(19) United States
(12) Patent Application Publication
(10) Pub. No.: US 2021/0322828 A1
(43) Pub. Date: Oct. 21, 2021

(54) Methods and Systems for Targeted Exercise Programs and Content

(71) Applicant: SPINE PRINCIPLES LLC, Malibu, CA (US)

(72) Inventor: Ezequiel Dario Gherscovici, Los Angeles, CA (US)

(21) Appl. No.: 17/235,658

(22) Filed: Apr. 20, 2021

Related U.S. Application Data
(60) Provisional application No. 63/012,775, filed on Apr. 20, 2020.

(51) Int. Cl. A63B 24/00; A63B 71/06; A61B 5/11; G16H 20/30

(52) U.S. Cl. CPC A63B 24/0075; A63B 71/0622; A61B 5/1118; A63B 2220/56; A63B 2071/0675; A63B 2220/836; A63B 2220/62; G16H 20/30

(57) ABSTRACT

Systems, devices, and methods including a set of sensors configured to detect and measure physical properties in the form of a sensor data and transmit the sensor data to an external computing device; and the external computing device having a processor configured to: receive the sensor data; determine a set of one or more preventative exercises and one or more rehabilitative exercises for a user; determine a set of media content, where the determined set of media content includes one or more actions to be performed by a user; receive the sensor data and the user input corresponding to the one or more actions by the user; and determine one or more new actions to be performed by the user based on the received continual sensor data and the user input.

### CROSS-REFERENCE TO RELATED APPLICATIONS

This application claims priority to and the benefit of U.S. Provisional Patent Application No. 63/012,775, filed Apr. 20, 2020, the contents of which are hereby incorporated by reference herein for all purposes.

### TECHNICAL FIELD

Embodiments relate generally to targeted exercise programs and content, and more particularly to dynamically updating a user's targeted exercise program and content based on the user's feedback.

### BACKGROUND

Prevention exercise programs, such as instructional neck and/or spine prevention or rehabilitation audio and video programs allow a user of the program to try and take control of their pain. A user's pain can be dynamic, and each user responds to prevention and rehabilitation exercises differently. Existing prevention and rehabilitation programs are static and do not consider the differences in user response and/or user pain levels.

### SUMMARY

Systems and methods may include dynamically providing targeted exercise programs and content to a user. The disclosed systems and methods provide a customized and targeted prevention exercise program/series selected for the end user based on previous feedback provided while performing the exercises and on the new task/new exercises the end user is planning to do. While performing the new task, the customized and targeted prevention program/series is constantly updated based on the end user's current and new feedback.

The customized and targeted prevention exercise programs/series may be selected by a computing device for the user based on user input, such as previous feedback provided by the user at a pop-up window of the computing device while performing the exercise and on new exercises the user is planning to perform. In one embodiment, while the user performs a new exercise, the customized and targeted prevention program/series may be constantly updated by the computing device based on the user's current and new feedback. In one embodiment, the user's customized and targeted exercise program includes a customized and targeted neck and/or spine prevention or rehabilitation program. In one embodiment, the program may be dynamically updated in real-time based on the user's previous, current, and/or new feedback. In one embodiment, a machine learning algorithm or the like may be used to continually refine the program based on the user's (and perhaps other user's) feedback history.

In one embodiment, a user may perform an exercise, after which the user is prompted at the computing device with a question related to the exercise. Based on the user's answer, the computing device may suggest a new exercise, a follow-up exercise, or other courses of action. In another embodiment, the user may be prompted with a question during the exercise. Based on the user's answer, the computing device may suggest a new exercise or other course of action. As such, the user's customized and targeted exercise program, such as a customized and targeted neck and/or spine prevention or rehabilitation program may be dynamically updated in real-time based on user's previous, current, and/or new feedback. In one embodiment, a machine learning algorithm or the like may be used to tailor the program based on the user's (and perhaps other user's) history.

A system embodiment may include a computing device with a processor and addressable memory, where the processor is configured to: receive media content, wherein the media content may include preventative and/or rehabilitative exercises for a user; extract a portion of the media content including an action to be performed by a user; prompt the user with the action to be performed; receive input after or during performing of the action by the user; and select a new action to be performed by the user based on the received input. The preventative and/or rehabilitative exercises may be customized and targeted for the user and may be constantly updated based on the user's feedback.

A system embodiment may also include an online self-directed educational platform utilizing engineering direct response marketing that creates targeted lead generation and sales conversions re-directing prospects to targeted exercise educational programs through artificial intelligence (AI) integration.

A system embodiment may include: a set of one or more sensors configured to detect and measure physical properties in the form of a sensor data and transmit the sensor data to an external computing device; and the external computing device having a processor and addressable memory, the processor configured to: receive the sensor data, where the sensor data includes at least one of: a behavioral data, a physical health data, a mental health data, a biomechanical data, and a biomedical related data; determine a set of one or more preventative exercises and one or more rehabilitative exercises for a user based on the received sensor data and a user input; determine a set of media content associated with the determined set of one or more preventative exercises and one or more rehabilitative exercises, where the determined set of media content includes one or more actions to be performed by a user; receive the sensor data and the user input corresponding to the one or more actions by the user, where the sensor data may be collected continually over a period of time; and determine one or more new actions to be performed by the user based on the received continual sensor data and the user input.

In additional system embodiments, the user input may be received in a time period comprising at least one of: before the user performs any action of the one or more actions, during performance of an action of the one or more actions by the user, and after the performance of an action of the one or more actions by the user. In additional system embodiments, the received sensor data creates a baseline of use by the user based on a time period the received sensor data was collected, and where the time period may be at least one of: during a pain-free stage, during a compliance with the determined one or more actions, and a post completion of the determined one or more actions. In additional system embodiments, the set of one or more sensors track a physical activity of the user using wearable devices. In additional system embodiments, the wearable devices include sensors to collect biomechanical alignments, misalignments, and biomedical metrics. In additional system embodiments, the set of one or more sensors may be placed in two or more furniture items to collect data associated with usage of the two or more furniture items. In additional system embodiments, the data collected includes a time and a frequency spent on each of the two or more furniture items. In additional system embodiments, the set of one or more sensors placed in the two or more furniture items measure a pressure placed on the two or more furniture items by the user and monitor a frequency of use of the two or more furniture items by the user.

A method embodiment may include: detecting and measuring, by a set of one or more sensors, physical properties in the form of a sensor data; transmitting, by the set of one or more sensors, the sensor data to an external computing device having a processor and addressable memory; receiving, by the processor, the sensor data, where the sensor data includes at least one of: a behavioral data, a physical health data, a mental health data, a biomechanical data, and a biomedical related data; determining, by the processor, a set of one or more preventative exercises and one or more rehabilitative exercises for a user based on the received sensor data and a user input; determining, by the processor, a set of media content associated with the determined set of one or more preventative exercises and one or more rehabilitative exercises, where the determined set of media content includes one or more actions to be performed by a user; receiving, by the processor, the sensor data and the user input corresponding to the one or more actions by the user, where the sensor data may be collected continually over a period of time; and determining, by the processor, one or more new actions to be performed by the user based on the received continual sensor data and the user input.

In additional method embodiments, the user input may be received in a time period comprising at least one of: before the user performs any action of the one or more actions, during performance of an action of the one or more actions by the user, and after the performance of an action of the one or more actions by the user. Additional method embodiments may include: creating, by the processor, a baseline of use by the user from the received sensor data based on a time period the received sensor data was collected, and where the time period may be at least one of: during a pain-free stage, during a compliance with the determined one or more actions, and a post completion of the determined one or more actions. Additional method embodiments may include: tracking, by the set of one or more sensors, a physical activity of the user using wearable devices. In additional method embodiments, the wearable devices include sensors to collect biomechanical alignments, misalignments, and biomedical metrics.

In additional method embodiments, the set of one or more sensors may be placed in two or more furniture items to collect data associated with usage of the two or more furniture items. In additional method embodiments, the data collected includes a time and a frequency spent on each of the two or more furniture items. Additional method embodiments may include: measuring, by the set of one or more sensors placed in the two or more furniture items, a pressure placed on the two or more furniture items by the user; and monitoring, by the set of one or more sensors placed in the two or more furniture items, a frequency of use of the two or more furniture items by the user.

A computing device embodiment may have a processor and addressable memory configured to: obtain a user input data from one or more sensors; extract a pain intensity data from the obtained user input data; determine if a predetermined threshold has been met based on the extracted pain intensity data; provide an alert if the predetermined threshold has been met; extract a furniture use data from the user input data; and map the pain intensity data to the extracted furniture use data.

In additional device embodiments, the furniture use data comprises data associated with a usage of two or more furniture items by a user, a pressure placed on the two or more furniture items by the user, and a frequency of use of the two or more furniture items by the user. In additional device embodiments, the processor may be further configured to: prepare one or more pain preventing strategies based on the mapped data. In additional device embodiments, the processor may be further configured to: map the pain intensity data to at least one of: biomechanical misalignments, biomedical markers, behavioral changes, and indoor environment designs.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 is a functional block diagram depicting an exemplary process of a computing device for processing of exercise media content;

FIG. 2 is an exemplary top-level functional block diagram of a computing device embodiment displaying exercise media content;

FIG. 3 is a flowchart of a method for dynamically updating a user's exercise program and content in real-time based on the user's feedback

FIG. 4 illustrates an example top-level functional block diagram of a computing device embodiment;

FIG. 5 shows a high-level block diagram and process of a computing system for implementing an embodiment of the system and process;

FIG. 6 shows a block diagram and process of an exemplary system in which an embodiment may be implemented;

FIG. 7 depicts a cloud computing environment for implementing an embodiment of the system and process disclosed herein;

FIG. 8 depicts the positioning of a set of sensors in relation to a user, according to an embodiment of this disclosure;

FIG. 9 depicts the positioning of a set of sensors in a room, according to an embodiment of this disclosure;

FIG. 10 shows a functional block diagram where a platform is receiving data from a set of sensors, according to an embodiment of this disclosure;

FIG. 11 depicts the user input data from the set of sensors being transmitted to a platform;

FIG. 12 is a flowchart of a method for monitoring a user's physical activity for pain; and

FIG. 13 is a flowchart of a method for determining actions for a user based on continually received sensor data and user input.

### DETAILED DESCRIPTION

The described technology concerns one or more methods, systems, apparatuses, and mediums storing processor-executable process steps to dynamically provide targeted exercise programs and content to a user. The disclosed systems and methods provide a customized and targeted prevention exercise program/series selected for the end user based on previous feedback provided while performing the exercises that help the user take control of their pain and on the new task/new exercises the end user is planning to do. While performing the new task, the customized and targeted prevention program/series is constantly being updated based on the end user's current and new feedback.

In one embodiment, the customized and targeted prevention exercise programs/series may be selected by a computing device for the user based on user input, such as previous feedback provided by the user at a pop-up window of the computing device while performing the exercise and on new exercises the user is planning to perform. In one embodiment, while the user performs a new exercise, the customized and targeted prevention program/series may be constantly updated by the computing device based on the user's current and new feedback. In one embodiment, the user's customized and targeted exercise program includes a customized and targeted neck and/or spine prevention or rehabilitation program. In one embodiment, the program may be dynamically updated in real-time based on the user's previous, current, and/or new feedback. In one embodiment, a machine learning algorithm or the like may be used to continually refine the program based on the user's (and perhaps other user's) feedback history.

In one embodiment, a user may perform an exercise, after which the user is prompted at the computing device with a question related to the exercise. Based on the user's answer, the computing device may suggest a new exercise, a follow-up exercise, or other courses of action. In another embodiment, the user may be prompted with a question during the exercise. Based on the user's answer, the computing device may suggest a new exercise or other course of action. As such, the user's customized and targeted exercise program, such as a customized and targeted neck and/or spine prevention or rehabilitation program may be dynamically updated in real-time based on user's, previous, current, and/or new feedback. In one embodiment, a machine learning algorithm or the like may be used to tailor the program based on the user's (and perhaps other user's) history.

In one embodiment, a user may be monitored by sensors to track the user's physical activity. That is, a sensor device which may be configured to detect or measures a physical property and record, indicate, or otherwise respond to the detected physical property in the form of sensor data. The sensor data, the data obtained by the sensors, may be utilized to provide the user with an exercise program. Such sensor data may be collected periodically on a continual basis and transmitted to a central server at regular intervals or random intervals. The sensors may be configured to detect events or changes in their environment and transmit the information to other electronic devices, frequently a computer processor, or may have an addressable memory in which the sensor data may be stored and released and/or transmitted at time intervals different than the time intervals used for collecting the data.

In one embodiment, wearables having sensors may be used to provide a means for data collection, monitoring, and measuring user/patients' daily activities to prevent new episodes of back pain or early detection of back pain onset and rapidly respond with effective strategies. By the proliferation of the Internet of Things (IoT) devices, in the form of sensors, coupled with Artificial Intelligence, the system may be configured to measure pressure, monitor time and frequency of use of users' furniture in their workplace environment, as well as in their home. These sensors may be placed in chairs, sofas, and mattresses to collect data before, during, and after a diagnosis is made or is necessary. In one embodiment, the sensors may communicate with each other before transmitting any information to a computing device, generating new data regarding user/patient's daily activity and new information with dataset may be generated to develop behavioral changes to prevent back pain and/or effectively manage acute episodes of back pain. In one example, the data may be gathered during a period where the user is not yet a patient and not experiencing any pains but allow the system to accumulate reference data associated with that user as far as their behavior and habits may be concerned. That is, the system may be configured to collect and provide prevention data, where the user/patient is pain-free. The sensors may collect such data and create a baseline of use or mapping, where the data may include time and frequency spent on each piece of furniture by the user as monitored and measured. Wearables may also be used by the user/patient to monitor and measure biomechanical alignments, misalignments as well as biomedical metrics.

Based on the collected data during a pain-free stage of a user, the system may determine a diagnosis and data correlation with more accuracy when a user/patient develops any pains, e.g., back pain. The system may, by way of the sensors, create a new baseline of use or painful mapping. That is, time and frequency spent on each piece of furniture by the user may be identified as well as when the pain started after using the previously indicated furniture. Data may be collected and new information and insights may be generated by the system, indicating a possible reduction in time and frequency on specific furniture to avoid back pain onset. In such embodiments, wearables may be used to identify biomechanical misalignments, correlation with pain onset, and changes in biomedical metrics.

In another embodiment, behavioral changes may be tracked by the system, where the sensors within the furniture may identify the user/patient while using the furniture and as previously collected. Additionally, the sensor may collect information and allow the system to identify when it is appropriate to change positions in order to avoid developing an onset of back pain by reducing time and frequency of use. The user/patient may be informed by vibration and through verbal language communication. New data may be collected, and new predictions generated, providing new prevention strategies. Through wearables, biomechanical misalignments, biomedical markers, as well as behavioral changes coupled with indoor environmental designs may all be closely monitored and measured during the three stages and correlate them with back pain onset.

A system embodiment may also include an online self-directed educational platform utilizing engineering direct response marketing that creates targeted lead generation and sales conversions re-directing prospects to targeted exercise educational programs through artificial intelligence (AI) integration.

A system embodiment may also include a compliance component that checks for compliance with a current treatment plan. The compliance component accomplishes this by analyzing biometric information provided by a patient and associating an emotional status with the patient. The biometric information provided by a patient may include voice, body gestures, facial patterns, and the like. A healthcare provider can be provided with the associated emotional status and determine whether a patient is complying with a treatment plan and if changes to the treatment plan are necessary to increase the level of compliance. A system embodiment may also include the use of a cybersecurity framework to protect user privacy and data. That is, some or all of the data collected and/or transmitted may be encrypted by the transmitting device in order to protect user data and, in turn, patient information. The techniques introduced below may be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), etc.

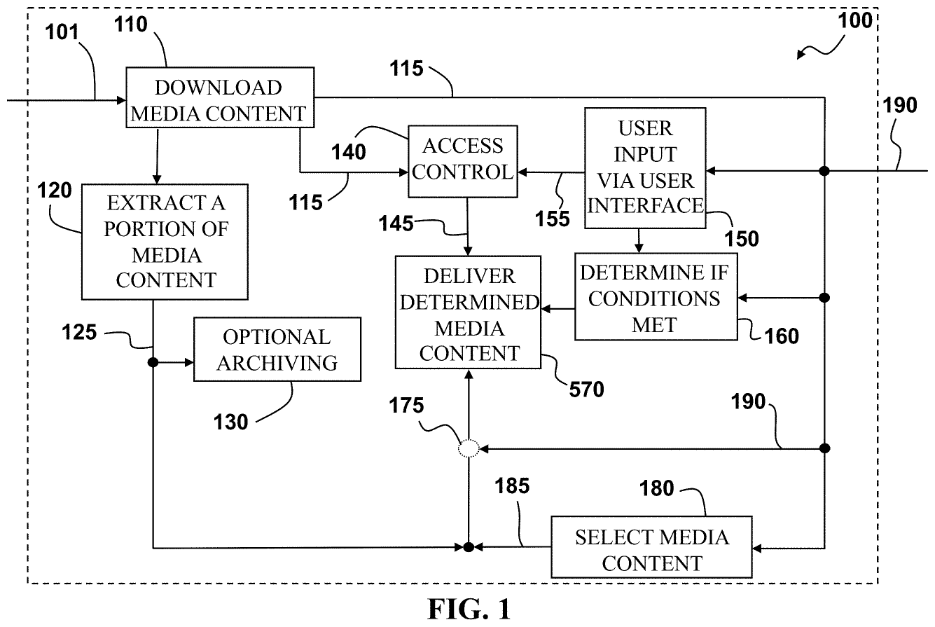

FIGS. 1-13 and the following discussion provide a brief, general description of a suitable computing environment in which aspects of the described technology may be implemented. Although not required, aspects of the technology may be described herein in the general context of computer-executable instructions, such as routines executed by a general- or special-purpose data processing device (e.g., a server or client computer). Aspects of the technology described herein may be stored or distributed on tangible computer-readable media, including magnetically or optically readable computer discs, hard-wired or preprogrammed chips (e.g., EEPROM semiconductor chips), nanotechnology memory, biological memory, or other data storage media. Alternatively, computer-implemented instructions, data structures, screen displays, and other data related to the technology may be distributed over the Internet or over other networks (including wireless networks) on a propagated signal on a propagation medium (e.g., an electromagnetic wave, a sound wave, etc.) over a period of time. In some implementations, the data may be provided on any analog or digital network (e.g., packet-switched, circuit-switched, or other scheme).

Present embodiments provide for dynamically updating a user's exercise program and content based on the user's feedback. In one embodiment of the system of the present disclosure, a user may perform an exercise, after which (or during which) the user may be prompted at a computing device with a question related to the exercise, such as pain level, range of motion, and the like. Based on the user's answer, the computing device may suggest a new exercise, subsequent exercise, or other courses of action. As such, the user does not follow a static, predetermined exercise protocol for prevention and/or rehabilitation; rather, the exercise program and other content provided to the user may be updated in real-time based on the user's set of previous responses to questions related at least to the exercise being performed or the exercise that was just completed. Additionally, prior routines may be used as a way to determine the user's particular behaviors and ability to do the movements, thereby determining an effectiveness level for each exercise that is being proposed. In one embodiment, sensors may be placed on the user's body, providing more detailed information to the computing device and therefore enhancing the ability of the computing device to provide further instructions and provide subsequent exercises in order to help the user achieve a particular goal. In one embodiment, the computing device may send notifications to the user before, during, and/or after the exercise. In one embodiment, the notification may be an alarm notification, such as a text notification in a pop-up window or an audible alarm to take an action. In one embodiment, the action may be providing feedback by the user to a question prompted to the user at the computing device. In another embodiment, the notification may be a timer indicating a time remaining for an exercise to be performed by the user. In another embodiment, the notifications may be sent by the system to a healthcare provider (via their user interface or other means disclosed herein) to notify them of changes while a patient is performing exercises. Such changes may be determined using the sensor data readings or other methods disclosed and include, for example, a change to a previous diagnosis, change to a previous classification syndrome, change to a previous sub-classification based on pain pattern responses, change in a pain intensity level, change in a pain location, change in a type of pain, change in a range of motion, change in a difficulty of performing an activity, change in fear or confidence to perform a movement, an activity or a job, change in mood, change in strength, change in sensitivity, change in sleep pattern, and/or change in appetite.

FIG. 1 depicts a functional block diagram of an exemplary computing device 100. In one embodiment, media content 101 may be received, e.g., downloaded 110 to the computing device 100. In one embodiment, the media content may include preventative and/or rehabilitative exercises for a user, such as a user with head and/or neck pain. In another embodiment, the media content may be an instruction or list of instructions for how to perform an exercise. Once the content is downloaded, the downloaded media content may be categorized where a portion of the media content 125 may be extracted 120, and optionally, the extracted portion 125 may be sent to an archiving component 130 so that the data is stored for future use, for example, future access to the content. In one embodiment, the portion of the media content may be a video of an exercise extracted from a library of videos of various exercises. Additionally, content, for example, the extracted portion of the downloaded media content 125 may be combined with other content, such as showing more than one exercise video. Therefore, a user may access content or re-access content at a later time.

The computing device 100 may also receive as input, user data 190, corresponding to a user. In one embodiment, the computing device 100 receives an initial questionnaire filled out by the user. The computing device 100 may then provide media content, such as exercises to perform, based on the user's answers in the initial questionnaire. The user may receive media content, such as a plurality of exercises to perform in the form of video segments or in the form of written instructions. In one embodiment, the user may then be prompted, such as with a pop-up window with a question related to the exercise performed. For example, the user may be asked, “was the pain getting (1) worse, (2) better, or (3) stayed the same?” In another embodiment, the user may then be prompted, such as with a pop-up window with a question related to the exercise being performed. For example, “Is the range of motion (1) getting bigger (2) getting smaller, or (3) not changing?” In another embodiment, the questionnaire may be used for diagnostic purposes. The results of the questionnaire may be compared to a diagnostic template to provide a provisional diagnosis. The provisional diagnosis may have a corresponding treatment plan and may be confirmed by an expert in the field. The expert in the field may adjust the diagnostic template to further optimize the diagnostic process. In one embodiment, the computing device may send notifications to the user before, during, and/or after the exercise. In one embodiment, the notification may be an alarm notification to take an action. The alarm may be a text notification displayed at the computing device. In one embodiment, the alarm may be an audible noise provided to the user by the computing device. In one embodiment, the action may be providing feedback by the user to a question prompted to the user at the pop-up window of the computing device, such as the question described above. In another embodiment, the notification may be a timer indicating the time remaining for an exercise to be performed by the user. In another embodiment, the notification may be a timer indicating the time remaining for the user to respond to the question in the pop-up window. The computing device may then tailor the exercise program, such as the next exercise to be provided to the user, based on the responses provided by the user.

The user may then provide feedback or input 190 after or while performing the exercises. In another embodiment, the user may provide input 190 based on new exercises the user is planning to perform. In one embodiment, while the user performs a new exercise, the computing device 100 may constantly update the customized and targeted prevention program/series based on the user's current and new input 190. In some embodiments, the user input data 190 may be received via a user interface 150, from a remote server, or previously stored on the computing device 100. In another embodiment, the user input data may be obtained from a set of sensors placed on the body of the user. The user data 190 may also be used to determine if certain conditions have been met 160, where the conditions may be based on the response to the prompted user response from the pop-up window, previous user inputs, and likely—predicted—future user inputs. An access control 140 may take as input a set of one or more of the following: downloaded media content 115 and user input data 155. In some embodiments, user data 190 may also be used to select 180, by the computing device 100, a next video segment or set of instructions from the received or downloaded media content 115. In one embodiment, once access to media content has been determined 145, the determined media content based on the user data 190 may be delivered to the user.

In one embodiment, the user receives a video segment or set of instructions for an exercise based on the answer provided in the user response data 190. For example, if the user selected option (1), then the computing device 100 selects 180 a specific video segment or set of instructions based on the selected option (1). If the user selected option (2), then the computing device 100 selects 180 a different video segment or set of instructions based on the selected option (2), and so forth. As such, the exercise regimen is not pre-defined; rather, the regimen is dynamic and updates in real-time based on the user input data 190. Optionally, modified media content 185 may then be combined 175 with user data 190 and provided as input to determine what media content may be delivered 170 to a display device. In one embodiment, once access to media content has been determined 145, the determined media content may be delivered 170 along with any modified content 180 that may have been determined based on the user data 190. In one embodiment, the computing system 100 may learn how to predict and provide tailored exercise regimens based on a user's input data history, such as with a machine learning algorithm. In one embodiment, the computing system 100 may provide for data mining of outside media content related to exercise and rehabilitation videos, such as new movements determined to be helpful in the scientific literature. The computing device 100 may add said outside media content to the computing device's 100 library of media content.

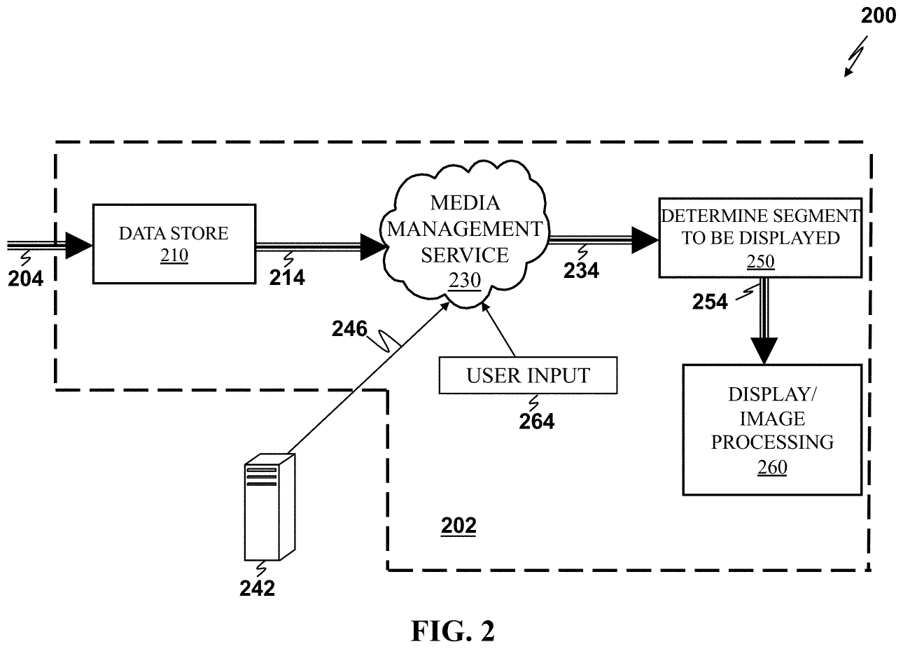

FIG. 2 depicts a block diagram of an exemplary embodiment of a media management service 230 of an access control controller 200. In this embodiment, media content 204 may be received by a computing device 202 where the computing device 202 optionally comprises a data store 210 and where the data store 210 may store the received media content 204 or alternatively, a subset of the media content. In some embodiments, the media content 204 may comprise a set of one or more media contents 214, such as video segments or instructions of exercises to be performed by a user. The set of one or more media contents 214 may then be sent to a media management service 230, where the media management service 230 may be running on the computing device 202. In one embodiment, the media management service 230 may also receive as input a set of user inputs 264 and other user data 246 received from a server 242, such as the initial questionnaire and the user-prompted responses described above. That is, data may be compiled using methods such as by crowd-sourcing data from other users who have exhibited similar ailments or goals. Optionally, the server 242 may reside outside of the computing device 202. In one embodiment, the media management service 230 may identify which set of the set of one or more media content 214 may be provided to the user based on received user input 264, and user data 246. In one embodiment, a separate service 250 may determine what subsequent segment of media content 234 may be sent to the display, for example, which new video exercise or set of exercise instruction may be displayed on the screen for the user. A display/image processing unit 260 may then receive the determined set of the media content 254 that are to be displayed.

The described technology may also be practiced in distributed computing environments where tasks or modules are performed by remote processing devices, which are linked through a communications network, such as a Local Area Network (“LAN”), Wide Area Network (“WAN”), or the Internet. In a distributed computing environment, program modules or subroutines may be located in both local and remote memory storage devices. Those skilled in the relevant art will recognize that portions of the described technology may reside on a server computer, while corresponding portions may reside on a client computer (e.g., PC, mobile computer, tablet, or smart phone). Data structures and transmission of data particular to aspects of the technology are also encompassed within the scope of the described technology.

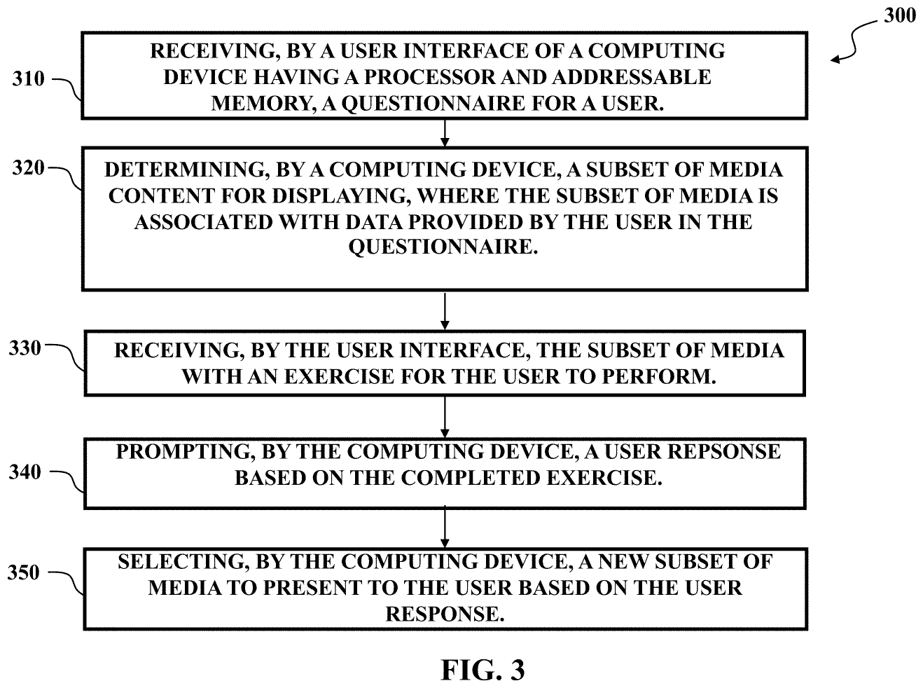

With respect to FIG. 3, a flowchart of a method 300 for dynamically updating a user's exercise program and content in real-time based on the user's feedback. At step 310, a user receives a questionnaire from a computing device having a processor and addressable memory. In one embodiment, the questionnaire contains questions related to a user's history with neck/and or back pain, as well as demographic questions. At step 320, the computing device may then determine a subset of media content for displaying to the user based on the answers provided by the user in the questionnaire. In one embodiment, the subset of media content may be a video segment of an exercise or a list of instructions for an exercise. At step 330, the subset of media may be received by the user interface. The user may perform the exercise after which, or during which, the user is prompted by the computing device to respond to a question based on the exercise performed or being performed, at step 340. For example, a question may be, “was the pain getting (1) worse, (2) better, or (3) stayed the same?” The user selects a response, either (1), (2), or (3), and the computing device, in turn, selects a new subset of media to present to the user based on the user response at step 350. For example, if the user selected option (1), then the computing device 100 selects 180 a specific video segment or set of instructions based on the selected option (1). If the user selected option (2), then the computing device 100 selects 180 a different video segment or set of instructions based on the selected option (2), and so forth. As such, the customized and targeted prevention exercise program may be selected by the computing device for the user based on previous feedback provided by the user while performing the exercises and on new exercises the user is planning to perform. In one embodiment, while the user performs a new exercise, the customized and targeted prevention program/series may be constantly updated based on the user's current and new feedback.

The disclosed system embodiments are configured to provide an implementation of supervised machine learning and multimodal language in order to provide healthcare professionals a better understanding of user compliance and understanding of the corresponding treatment principles. A multimodal language model may include the full complement of fundamental modes of communication, including depiction, description, and indexing, and the wider context in which utterances are constructed and interpreted. That is, through visual and acoustic signals, speaker and face identification, lip/mouth reading, and speech recognition, a linguistic message may be associated with a specific persons' emotional state. The system, via the disclosed sensors, may receive and collect such information in real-time, and predict the user's behavior to determine if the user will be compliant with the treatment provided. If not, the system may change how the treatment is being explained until he/she has the outcome expected to have proper compliance before discharging the patient.

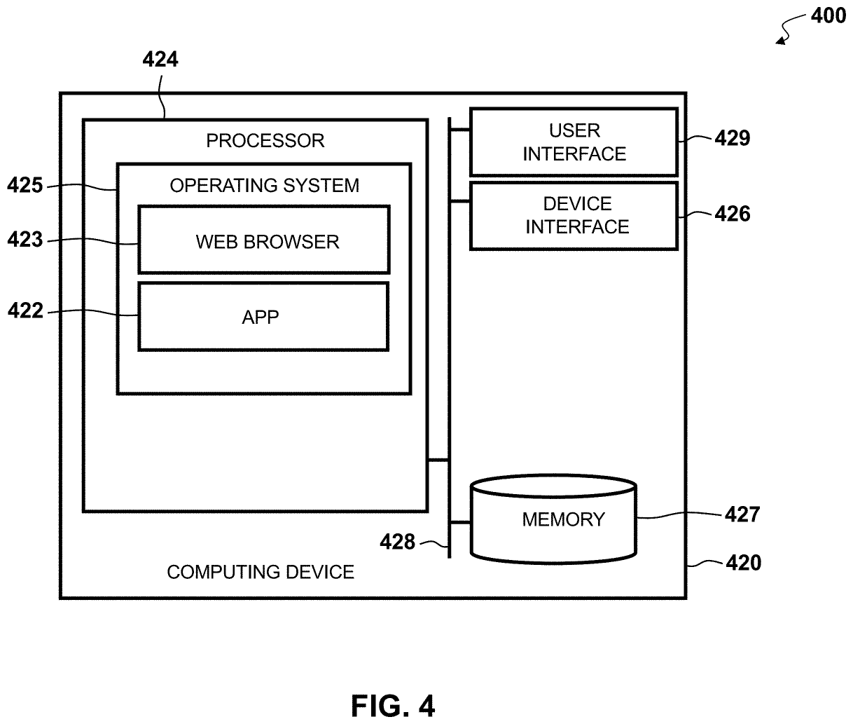

FIG. 4 illustrates an example of a top-level functional block diagram of a computing device embodiment 400. The example operating environment is shown as a computing device 420 comprising a processor 424, such as a central processing unit (CPU), addressable memory 427, an external device interface 426, e.g., an optional universal serial bus port and related processing, and/or an Ethernet port and related processing, and an optional user interface 429, e.g., an array of status lights and one or more toggle switches, and/or a display, and/or a keyboard and/or a pointer-mouse system and/or a touch screen. Optionally, the addressable memory may include any type of computer-readable media that can store data accessible by the computing device 420, such as magnetic hard and floppy disk drives, optical disk drives, magnetic cassettes, tape drives, flash memory cards, digital video disks (DVDs), Bernoulli cartridges, RAMs, ROMs, smart cards, etc. Indeed, any medium for storing or transmitting computer-readable instructions and data may be employed, including a connection port to or node on a network, such as a LAN, WAN, or the Internet. These elements may be in communication with one another via a data bus 428. In some embodiments, via an operating system 425 such as one supporting a web browser 423 and applications 422, the processor 424 may be configured to execute steps of a process establishing a communication channel and processing according to the embodiments described above.

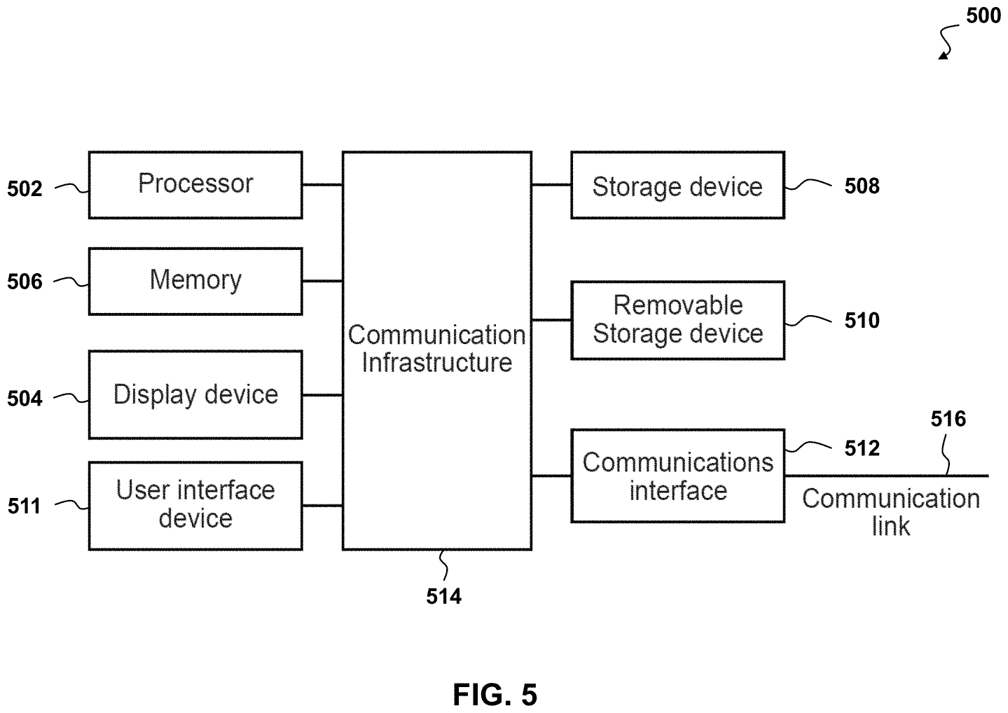

FIG. 5 is a high-level block diagram 500 showing a computing system comprising a computer system useful for implementing an embodiment of the system and process, disclosed herein. Embodiments of the system may be implemented in different computing environments. The computer system includes one or more processors 502, and can further include an electronic display device 504 (e.g., for displaying graphics, text, and other data), a main memory 506 (e.g., random access memory (RAM)), storage device 508, a removable storage device 510 (e.g., removable storage drive, a removable memory module, a magnetic tape drive, an optical disk drive, a computer readable medium having stored therein computer software and/or data), user interface device 511 (e.g., keyboard, touch screen, keypad, pointing device), and a communication interface 512 (e.g., modem, a network interface (such as an Ethernet card), a communications port, or a PCMCIA slot and card). The communication interface 512 allows software and data to be transferred between the computer system and external devices. The system further includes a communications infrastructure 514 (e.g., a communications bus, cross-over bar, or network) to which the aforementioned devices/modules are connected as shown.

Information transferred via communications interface 514 may be in the form of signals such as electronic, electromagnetic, optical, or other signals capable of being received by communications interface 514, via a communication link 516 that carries signals and may be implemented using wire or cable, fiber optics, a phone line, a cellular/mobile phone link, a radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer-implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc. Computer programs (i.e., computer control logic) are stored in main memory and/or secondary memory. Computer programs may also be received via a communications interface 512. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

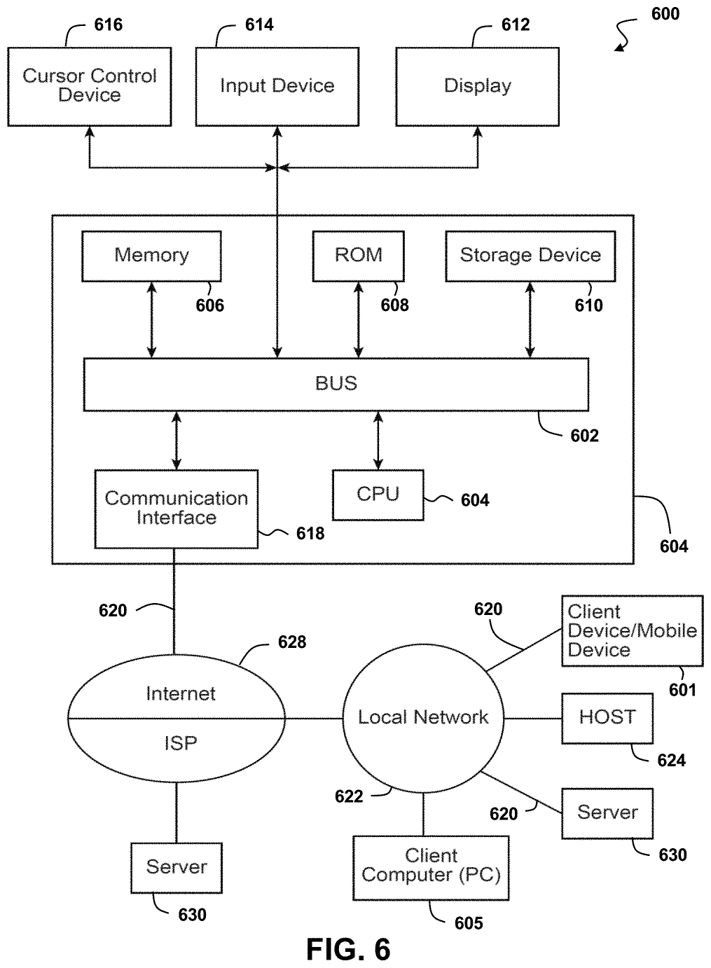

FIG. 6 shows a block diagram of an example system 600 in which an embodiment may be implemented. The system 600 includes one or more client devices 601, such as consumer electronics devices, connected to one or more server computing systems 630. A server 630 includes a bus 602 or other communication mechanism for communicating information, and a processor (CPU) 604 coupled with the bus 602 for processing information. The server 630 also includes a main memory 606, such as a random access memory (RAM) or other dynamic storage device, coupled to the bus 602 for storing information and instructions to be executed by the processor 604. The main memory 606 also may be used for storing temporary variables or other intermediate information during execution or instructions to be executed by the processor 604. The server computer system 630 further includes a read only memory (ROM) 608 or other static storage device coupled to the bus 602 for storing static information and instructions for the processor 604. A storage device 610, such as a magnetic disk or optical disk, is provided and coupled to the bus 602 for storing information and instructions. The bus 602 may contain, for example, thirty-two address lines for addressing video memory or main memory 606. The bus 602 can also include, for example, a 32-bit data bus for transferring data between and among the components, such as the CPU 604, the main memory 606, video memory and the storage 610. Alternatively, multiplex data/address lines may be used instead of separate data and address lines. The server 630 may be coupled via the bus 602 to a display 612 for displaying information to a computer user. An input device 614, including alphanumeric and other keys, is coupled to the bus 602 for communicating information and command selections to the processor 604. Another type or user input device comprises cursor control 616, such as a mouse, a trackball, or cursor direction keys for communicating direction information and command selections to the processor 604 and for controlling cursor movement on the display 612.

According to one embodiment, the functions are performed by the processor 604 executing one or more sequences of one or more instructions contained in the main memory 606. Such instructions may be read into the main memory 606 from another computer-readable medium, such as the storage device 610. Execution of the sequences of instructions contained in the main memory 606 causes the processor 604 to perform the process steps described herein. One or more processors in a multi-processing arrangement may also be employed to execute the sequences of instructions contained in the main memory 606. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 604 for execution. Such a medium may take many forms, including but not limited to, non-volatile media, volatile media, and transmission media. Non-volatile media includes, for example, optical or magnetic disks, such as the storage device 610. Volatile media includes dynamic memory, such as the main memory 606. Transmission media includes coaxial cables, copper wire and fiber optics, including the wires that comprise the bus 602. Transmission media can also take the form of acoustic or light waves, such as those generated during radio wave and infrared data communications. Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 604 for execution. For example, the instructions may initially be carried on a magnetic disk of a remote computer. The remote computer can load the instructions into its dynamic memory and send the instructions over a telephone line using a modem. A modem local to the server 630 can receive the data on the telephone line and use an infrared transmitter to convert the data to an infrared signal. An infrared detector coupled to the bus 602 can receive the data carried in the infrared signal and place the data on the bus 602. The bus 602 carries the data to the main memory 606, from which the processor 604 retrieves and executes the instructions. The instructions received from the main memory 606 may optionally be stored on the storage device 610 either before or after execution by the processor 604.

The server 630 also includes a communication interface 618 coupled to the bus 602. The communication interface 618 provides a two-way data communication coupling to a network link 620 that is connected to the world wide packet data communication network now commonly referred to as the Internet 628. The Internet 628 uses electrical, electromagnetic or optical signals that carry digital data streams. The signals through the various networks and the signals on the network link 620 and through the communication interface 618, which carry the digital data to and from the server 630, are exemplary forms or carrier waves transporting the information.

In another embodiment of the server 630, interface 618 is connected to a network 622 via a communication link 620. For example, the communication interface 618 may be an integrated services digital network (ISDN) card or a modem to provide a data communication connection to a corresponding type of telephone line, which can comprise part of the network link 620. As another example, the communication interface 618 may be a local area network (LAN) card to provide a data communication connection to a compatible LAN. Wireless links may also be implemented. In any such implementation, the communication interface 618 sends and receives electrical electromagnetic or optical signals that carry digital data streams representing various types of information.

The network link 620 typically provides data communication through one or more networks to other data devices. For example, the network link 620 may provide a connection through the local network 622 to a host computer 624 or to data equipment operated by an Internet Service Provider (ISP). The ISP in turn provides data communication services through the Internet 628. The local network 622 and the Internet 628 both use electrical, electromagnetic or optical signals that carry digital data streams. The signals through the various networks and the signals on the network link 620 and through the communication interface 618, which carry the digital data to and from the server 630, are exemplary forms or carrier waves transporting the information. The server 630 can send/receive messages and data, including e-mail, program code, through the network, the network link 620 and the communication interface 618. Further, the communication interface 618 can comprise a USB/Tuner and the network link 620 may be an antenna or cable for connecting the server 630 to a cable provider, satellite provider or other terrestrial transmission system for receiving messages, data and program code from another source.

The example versions of the embodiments described herein may be implemented as logical operations in a distributed processing system such as the system 600 including the servers 630. The logical operations of the embodiments may be implemented as a sequence of steps executing in the server 630, and as interconnected machine modules within the system 600. The implementation is a matter of choice and can depend on performance of the system 600 implementing the embodiments. As such, the logical operations constituting said example versions of the embodiments are referred to for e.g., as operations, steps or modules. Similar to a server 630 described above, a client device 601 can include a processor, memory, storage device, display, input device and communication interface (e.g., e-mail interface) for connecting the client device to the Internet 628, the ISP, or LAN 622, for communication with the servers 630. The system 600 can further include computers (e.g., personal computers, computing nodes) 605 operating in the same manner as client devices 601, where a user can utilize one or more computers 605 to manage data in the server 630.

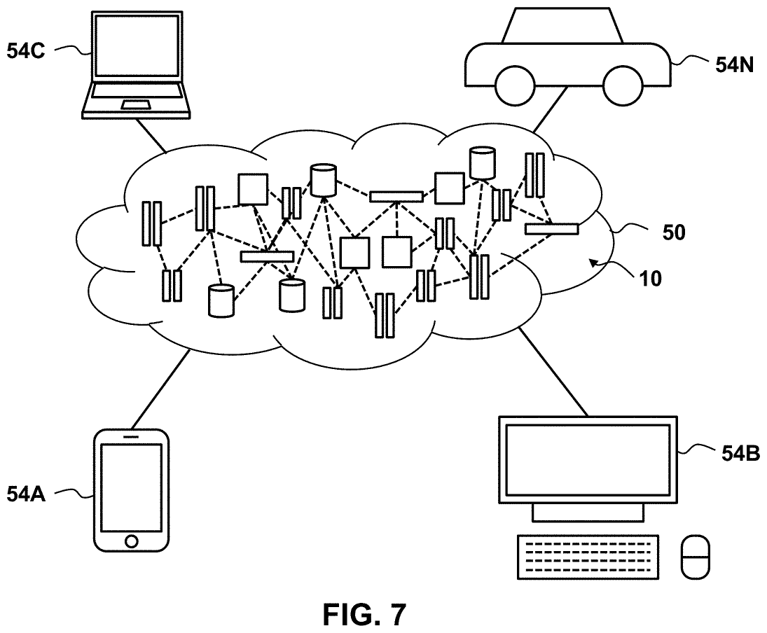

Referring now to FIG. 7, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 7 are intended to be illustrative only and that computing nodes 10 and cloud computing environment 50 can communicate with any type of computerized device over any type of network and/or network addressable connection (e.g., using a web browser).

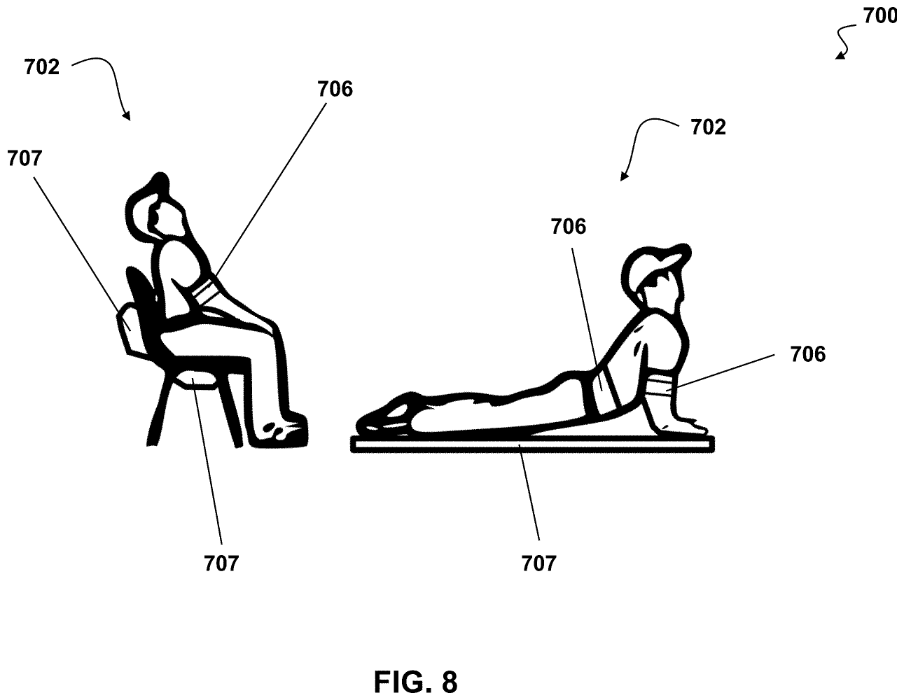

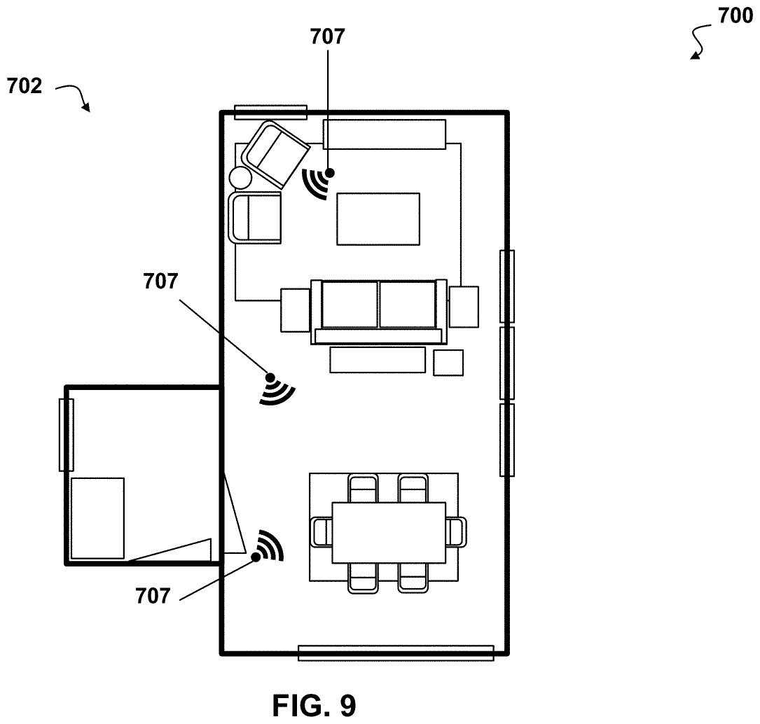

FIGS. 8-9 depict a system 700 with a set of sensors 702 for monitoring and collecting data related to a user and providing information on their health state, environment, etc. The set of sensors 702 may include a set of wearable sensors 706 and a set of non-wearable sensors 707. The set of wearable sensors 706 may be worn on a user's body and monitor the user's daily activity, heart rate, glucose levels, hydration levels, breathing frequency, blood pressure, pain intensity, posture, mood and behavioral changes, biometric information, and the like. The set of wearable sensors 706 may further monitor information associated with a specific exercise that is prescribed to a user. The set of wearable sensors 706 may be worn around a user's arm or around a user's waist. The set of wearable sensors 706 may have a vibration mechanism or a speaker to provide notifications. The non-wearable sensors 707 may be positioned in a living/working area and configured to monitor the physical activity near, on, or by a set of furniture over a period of time. The non-wearable sensors 707 may further be positioned directly on a piece of furniture to track physical activity by monitoring the pressure exerted on a piece of furniture over a period of time, length of time the furniture is used, time of day the furniture is used, and/or the manner in which the furniture is used (e.g., is the user laying down, raising their feet up, reclining, etc.). The set of wearable sensors 706 and the set of non-wearable sensors 707 may work in conjunction with each other to more accurately monitor information such as the posture of a user or a set of users. The sensors may be configured to detect a particular user amongst a set of users who may be using the furniture and associate the data collected with each user to determine behavioral changes needed and implemented by the user.

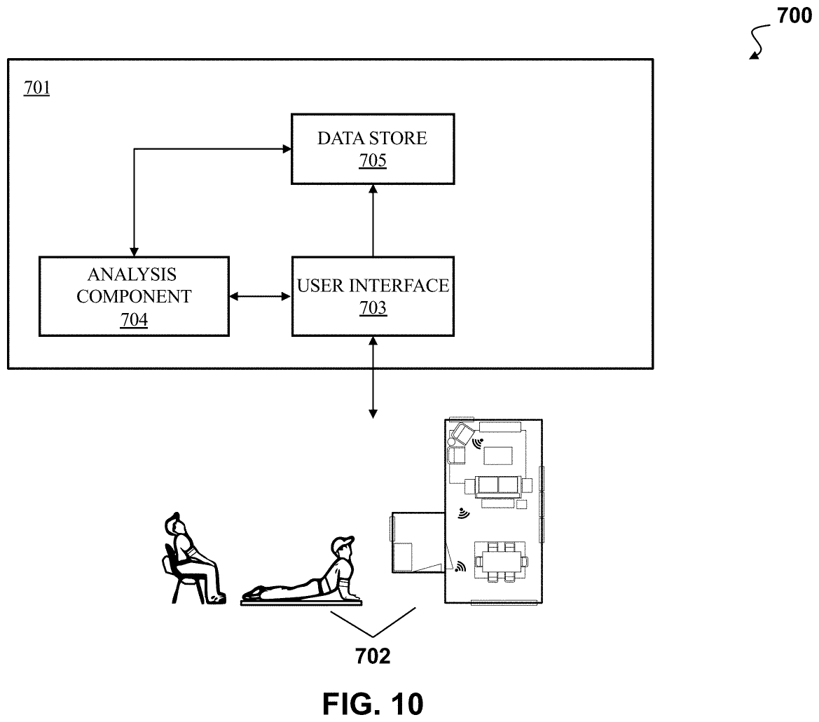

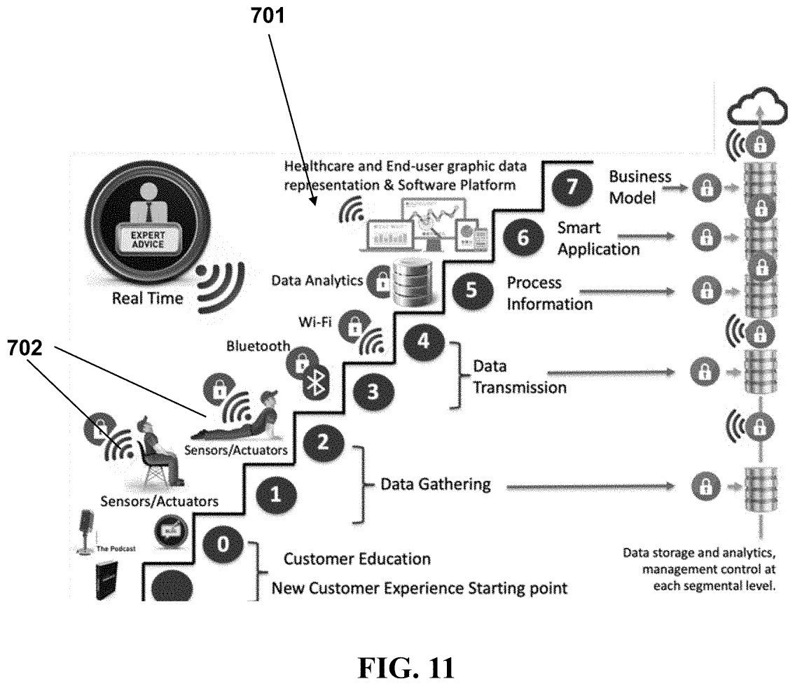

Referring to FIGS. 10-11, the system 700 further includes a platform 701 for receiving user input data from a set of sensors 702, according to an embodiment of this disclosure. The set of sensors 702 may send user input data to a user interface 703 using technologies such as Wi-Fi, Bluetooth, radio-frequency identification (RFID), wired connections, and the like. The user interface 703 may be configured to send the received user input data to an analysis component 704 and a data store 705. The analysis component 704 may rely on pre-existing templates stored in the data store 705 in combination with the received user input data to provide a user with information such as a provisional diagnosis or an exercise program. The analysis component 704 may be configured to use the received user input data to continuously update and optimize any pre-existing templates in the data store 705. The analysis component 704 may allow for experts to analyze the received user input data, and update and optimize any pre-existing templates in the data store 705.

The user interface 703 may provide a user with communication means for interacting with experts and fellow users. The communication means may include but is not limited to: video-conferencing, electronic messaging, short message service (SMS), and voice calls. The user interface 703 may facilitate ecommerce by providing a user with transaction means such as an e-payment system. The transaction data from the facilitated e-commerce may be sent by the user interface 703 to the analysis component 704 and the data store 705. The analysis component 704 may use the transaction data to update and optimize any pre-existing templates in the data store 705. The user interface 703 may provide a calendar to assist a user with setting up appointments with an expert.

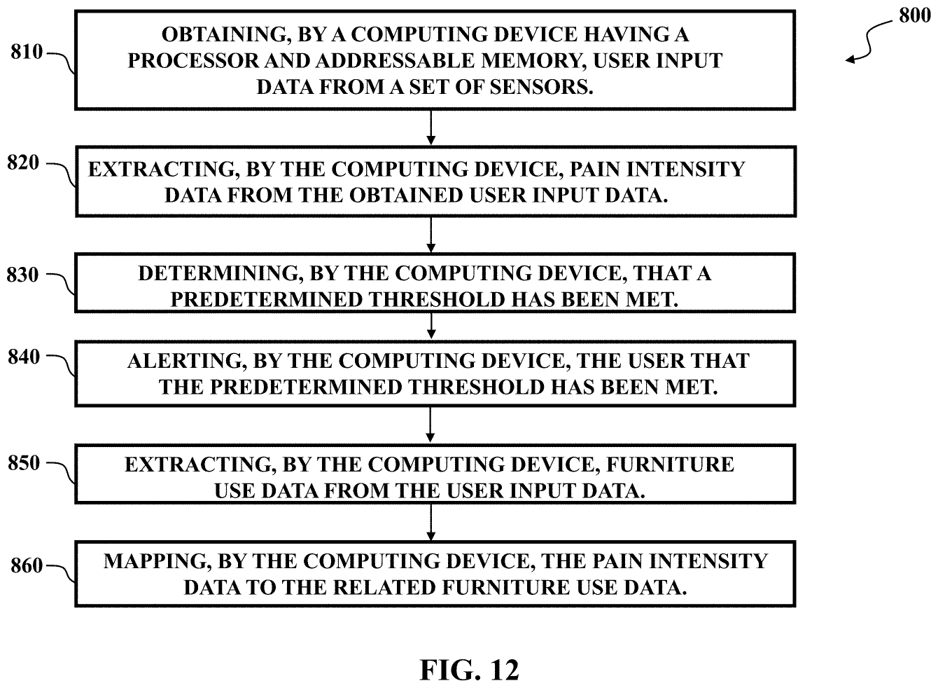

With respect to FIG. 12, a flowchart of a method 800 for monitoring the activity of a user for pain is illustrated, where the determined purpose may be to monitor the frequency of use, duration of use, and how often it is used and if there is any alteration of use when the patient is pain free or in pain and if in pain if there are any changes of use depending on the pain intensity or pain location. At step 810, a computing device with a processor and addressable memory may receive user input data from a set of sensors. At step 820, the computing device may be configured to extract pain intensity data from the obtained user input data. At step 830, the computing device may determine that the pain intensity data matches or exceeds a predetermined pain intensity threshold. By way of feedback previously obtained from the user/patients questionnaire, and as the sensors are recording data in real-time, and questions asked by the system, the system determines whether the paid is above a threshold. The computing device then alerts the user that the user's pain intensity level is above the predetermined pain intensity threshold, at step 840. At step 850, the computing device extracts furniture use data during the occurrence of physical pain from the user input data. The furniture use data may include the time and frequency that a user spends resting on furniture. In another embodiment, the furniture use data may be collected continuously and transmitted at a predefined time interval. At step 860, the computing device may map the pain intensity data with the related furniture use data. The mapping of pain intensity data to the furniture use data may assist in the preparation of new prevention strategies. In one embodiment, the pain intensity data is further mapped with biomechanical misalignments, biomedical markers, behavioral changes, and indoor environment designs that may improve performance and productivity.

The disclosed embodiments provide an automated self-directed educational system and platform that is updated in real-time or near real-time based on end user feedback and AI integration using exercises and prevention programs. These embodiments include computing devices configured to generate a provisional diagnosis of the current condition related to back/neck pain of a user (e.g., patient) prior to a visit to the healthcare provider. That is, health care providers may deliver care more rapidly and efficiently using the disclosed systems and methods. The system may employ machine learning (ML) to determine the frequency of notification to the user and determine a modification to the exercise program to adapt the exercise program to the user in real-time. The system may also transmit such notifications and determinations to the health care provider in order to allow them to make corrections and changes while the user (patient) is performing the pre-selected exercises in real-time. Continuous monitoring of the user-during recovery or post recovery—may be implemented to optimize and modify the frequency of notifications according to their individual needs. The above system may operate through artificial intelligence (AI) and its sub-set machine learning (ML) to reduce diagnostic error due to emotional distress or repetitive routine tasks associated with the daily routine healthcare providers are exposed to. Additionally, the system may assist health care providers in designing individualized matching treatment principles assigned to each patient, after the diagnosis is confirmed by the clinician.

One embodiment may utilize a statistical approach to providing diagnosis once the user provides answers to a series of questions. In such embodiments, the system may provide an automatic provisional diagnosis that may be generated with a set of associated treatment regiments, where a number of methods are present by providing comparisons between user provided answers to questions with previously provided answers by other users, for example, comparing user provided answers with a database of previously collected provisional diagnosis made based on answers provided by others in order to make a determination. The system also may optionally provide a “cyber human learning loop” where at a determined juncture, the system may request input from an expert to constantly update and continuously optimize the AI and ML models for better accuracy in the diagnosis.

The disclosed system may create an ongoing validation and retraining model by way of making individualized level predictions, where personalized content is delivered to the user. Such personalized content may be based on data collected about the user, and such data may include: data continuously collected through and via Internet of Things (IoT) devices, data received from a set of sensors collecting and transmitting data (e.g., pictures & video), language data (e.g., written and voice), and transaction data which may be used to update and optimize the system. Some embodiments may include classifications and labels leading to possible diagnosis based on stationary sensor data. Additionally, embodiments may provide temporal sensor data pattern recognition that may be used and identified, such as spatio-temporal patterns in sensor data. For example, temporal data collected may involve processing time series, typically sequences of data, which measure values of the same attribute at a sequence of different time points; such data may then be associated with a user. Another example may be pattern matching using such data, where the system may be configured to search for particular patterns of interest, also associated with a user and their behavior as it pertains to their health and more particularly, to their back or neck pain.

According to the disclosed embodiments, the system may be configured to both provide support to a healthcare provider while also providing a user interface to the user/patient. In one embodiment, the system aids the healthcare providers by determining a prediction of a possible diagnosis or classifications based on pain pattern behavior based on sensor data reading and processing. In this embodiment, the system may also provide, in real-time, a rehabilitation treatment plan and exercise prevention program. In yet another embodiment, the system may determine, for use by a healthcare professional, a user/patient's compliance to the treatment principles provided by the healthcare professional by monitoring and optimizing the diagnosis based on sensor data reading and processing. In an alternative embodiment, the system may be configured to provide and determine a set of instructions associated with the user to help reinforce behavior that contributes to empowering effective self-management of back and neck pain and diminishes detrimental behaviors that predispose back and neck pain onset.

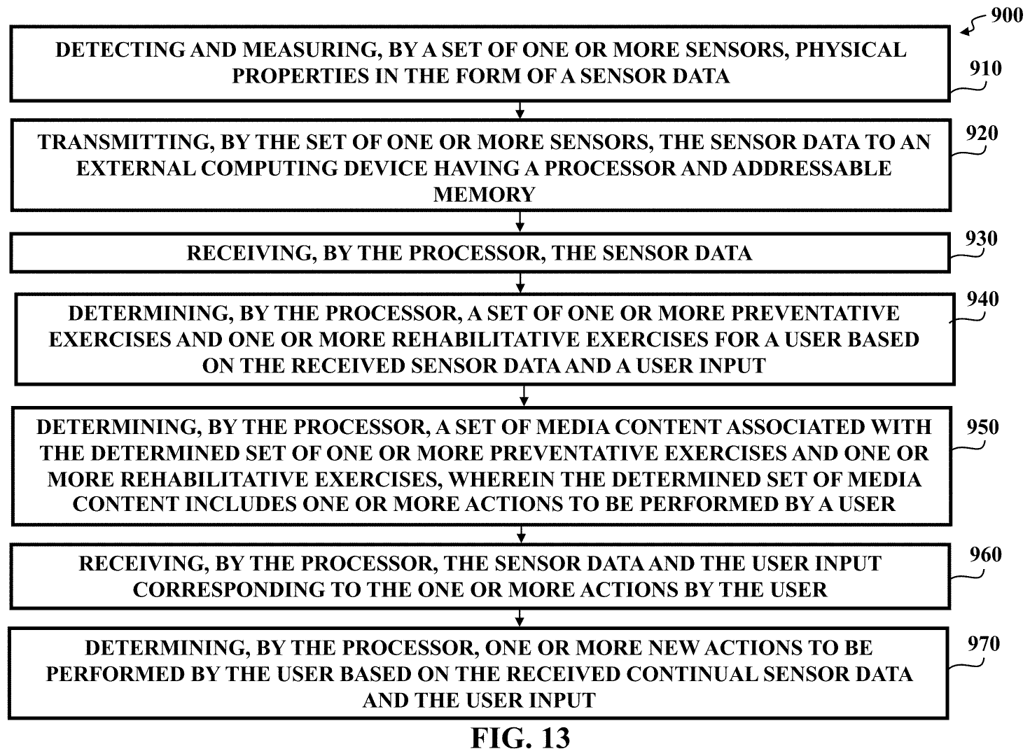

FIG. 13 is a flowchart of a method 900 for determining actions for a user based on continually received sensor data and user input. The method 900 may include detecting and measuring, by a set of one or more sensors, physical properties in the form of a sensor data (step 910). The method 900 may then include transmitting, by the set of one or more sensors, the sensor data to an external computing device having a processor and addressable memory (step 920). The method 900 may then include receiving, by the processor, the sensor data (step 930). The sensor data may include at least one of: a behavioral data, a physical health data, a mental health data, a biomechanical data, and a biomedical related data. The method 900 may then include determining, by the processor, a set of one or more preventative exercises and one or more rehabilitative exercises for a user based on the received sensor data and a user input (step 940). The method 900 may then include determining, by the processor, a set of media content associated with the determined set of one or more preventative exercises and one or more rehabilitative exercises, where the determined set of media content includes one or more actions to be performed by a user (step 950). The method 900 may then include receiving, by the processor, the sensor data and the user input corresponding to the one or more actions by the user (step 960). The sensor data may be collected continually over a period of time. The method 900 may then include determining, by the processor, one or more new actions to be performed by the user based on the received continual sensor data and the user input (step 970).

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention is herein disclosed by way of examples and should not be limited by the particular disclosed embodiments described above.

What is claimed is:

1. A system comprising:
a set of one or more sensors configured to detect and measure physical properties in the form of a sensor data and transmit the sensor data to an external computing device; and
the external computing device having a processor and addressable memory, the processor configured to:
receive the sensor data, wherein the sensor data includes at least one of: a behavioral data, a physical health data, a mental health data, a biomechanical data, and a biomedical related data;
determine a set of one or more preventative exercises and one or more rehabilitative exercises for a user based on the received sensor data and a user input;
determine a set of media content associated with the determined set of one or more preventative exercises and one or more rehabilitative exercises, wherein the determined set of media content includes one or more actions to be performed by a user;
receive the sensor data and the user input corresponding to the one or more actions by the user, wherein the sensor data is collected continually over a period of time; and
determine one or more new actions to be performed by the user based on the received continual sensor data and the user input.

2. The system of claim 1, wherein the user input is received in a time period comprising at least one of: before the user performs any action of the one or more actions, during performance of an action of the one or more actions by the user, and after the performance of an action of the one or more actions by the user.

3. The system of claim 1, wherein the received sensor data creates a baseline of use by the user based on a time period the received sensor data was collected, and wherein the time period is at least one of: during a pain-free stage, during a compliance with the determined one or more actions, and a post completion of the determined one or more actions.

4. The system of claim 1, wherein the set of one or more sensors track a physical activity of the user using wearable devices.

5. The system of claim 4, wherein the wearable devices include sensors to collect biomechanical alignments, misalignments, and biomedical metrics.

6. The system of claim 1, wherein the set of one or more sensors are placed in two or more furniture items to collect data associated with usage of the two or more furniture items.

7. The system of claim 6, wherein the data collected includes a time and a frequency spent on each of the two or more furniture items.

8. The system of claim 6, wherein the set of one or more sensors placed in the two or more furniture items measure a pressure placed on the two or more furniture items by the user and monitor a frequency of use of the two or more furniture items by the user.

9. A method comprising:
detecting and measuring, by a set of one or more sensors, physical properties in the form of a sensor data;
transmitting, by the set of one or more sensors, the sensor data to an external computing device having a processor and addressable memory;
receiving, by the processor, the sensor data, wherein the sensor data includes at least one of: a behavioral data, a physical health data, a mental health data, a biomechanical data, and a biomedical related data;
determining, by the processor, a set of one or more preventative exercises and one or more rehabilitative exercises for a user based on the received sensor data and a user input;
determining, by the processor, a set of media content associated with the determined set of one or more preventative exercises and one or more rehabilitative exercises, wherein the determined set of media content includes one or more actions to be performed by a user;
receiving, by the processor, the sensor data and the user input corresponding to the one or more actions by the user, wherein the sensor data is collected continually over a period of time; and
determining, by the processor, one or more new actions to be performed by the user based on the received continual sensor data and the user input.

10. The method of claim 9, wherein the user input is received in a time period comprising at least one of: before the user performs any action of the one or more actions, during performance of an action of the one or more actions by the user, and after the performance of an action of the one or more actions by the user.

11. The method of claim 9, further comprising:
creating, by the processor, a baseline of use by the user from the received sensor data based on a time period the received sensor data was collected, and wherein the time period is at least one of: during a pain-free stage, during a compliance with the determined one or more actions, and a post completion of the determined one or more actions.

12. The method of claim 9, further comprising: tracking, by the set of one or more sensors, a physical activity of the user using wearable devices.

13. The method of claim 12, wherein the wearable devices include sensors to collect biomechanical alignments, misalignments, and biomedical metrics.

14. The method of claim 9, wherein the set of one or more sensors are placed in two or more furniture items to collect data associated with usage of the two or more furniture items.

15. The method of claim 14, wherein the data collected includes a time and a frequency spent on each of the two or more furniture items.

16. The method of claim 14, further comprising:
measuring, by the set of one or more sensors placed in the two or more furniture items, a pressure placed on the two or more furniture items by the user; and
monitoring, by the set of one or more sensors placed in the two or more furniture items, a frequency of use of the two or more furniture items by the user.

17. A computing device having a processor and addressable memory configured to:
obtain a user input data from one or more sensors;
extract a pain intensity data from the obtained user input data;
determine if a predetermined threshold has been met based on the extracted pain intensity data;
provide an alert if the predetermined threshold has been met;
extract a furniture use data from the user input data; and
map the pain intensity data to the extracted furniture use data.

18. The device of claim 17, wherein the furniture use data comprises data associated with a usage of two or more furniture items by a user, a pressure placed on the two or more furniture items by the user, and a frequency of use of the two or more furniture items by the user.

19. The device of claim 17, wherein the processor is further configured to: prepare one or more pain preventing strategies based on the mapped data.

20. The device of claim 17, wherein the processor is further configured to:
map the pain intensity data to at least one of: biomechanical misalignments, biomedical markers, behavioral changes, and indoor environment designs.
